(19) United States
(12) Patent Application Publication
(10) Pub. No.: US 2018/0325548 A1
(43) Pub. Date: Nov. 15, 2018

(54) INTRODUCER WITH EXPANDABLE CAPABILITIES

(71) Applicant: BOSTON SCIENTIFIC SCIMED, INC., Maple Grove, MN (US)

(72) Inventors: Patrick A. Haverkost, Corcoran, MN (US); Timothy A. Ostroot, Cokato (MN); Cass Alexander Hanson, Saint Paul, MN (US); James M. Anderson, Corcoran, MN (US); David Raab, Roseville, MN (US)

(73) Assignee: BOSTON SCIENTIFIC SCIMED, INC., MAPLE GROVE, MN (US)

(21) Appl. No.: 15/979,098

(22) Filed: May 14, 2018

Related U.S. Application Data
(60) Provisional application No. 62/505,429, filed on May 12, 2017.

(51) Int. Cl. A61B 17/34; A61F 2/24

(52) U.S. Cl. CPC A61B 17/3468; A61B 17/3462; A61M 25/0054; A61B 17/3423; A61F 2/2427

(57) ABSTRACT

An example introducer is disclosed. An example introducer sheath includes a liner having a proximal portion, a distal portion and an axial slot extending along a portion thereof. The introducer sheath may also include a plurality of expandable members disposed adjacent at least a portion of the axial slot and a sheath disposed over at least a portion of the liner and the plurality of expandable members. Further, the introducer sheath is designed to shift between a first configuration and an expanded configuration

### CROSS-REFERENCE TO RELATED APPLICATIONS

This application claims the benefit of priority under 35 U.S.C. § 119 to U.S. Provisional Application Ser. No. 62/505,429, filed May 12, 2018, the entirety of which is incorporated herein by reference.

### TECHNICAL FIELD

The disclosure relates generally to medical devices and more particularly to medical devices that are adapted for use in percutaneous medical procedures.

### BACKGROUND

In some instances, performing percutaneous medical procedures may require the insertion and/or maneuvering of relatively large medical devices through a patient's vasculature. However, inserting the medical device into the vasculature may result in undesirable forces being applied to the vessel walls. For example, as the medical device passes into the vasculature, it may make undesirable contact with one or more vessel walls. This interference may cause injury to the vessel as the medical device is navigated into calcified or diseased vessels. Therefore, in some instances an introducer is utilize to facilitate the insertion of medical devices into the vessel. Further, vessel trauma resulting from forces applied to the vessel wall by a medical device may be lessened by minimizing the size of an introducer used to access the vessel. Therefore, it may be desirable to design an introducer having a reduced insertion profile, yet capable of expansion when necessary (e.g., during the passage of a medical device therethrough).

### SUMMARY

This disclosure provides design, material, manufacturing method, and use alternatives for medical devices. An example introducer sheath includes a liner having a proximal portion, a distal portion and an axial slot extending along a portion thereof. The introducer sheath may also include a plurality of expandable members disposed adjacent at least a portion of the axial slot and a sheath disposed over at least a portion of the liner and the plurality of expandable members. Further, the introducer sheath is designed to shift between a first configuration and an expanded configuration.

Alternatively or additionally to any of the embodiments above, wherein at least one of the plurality of expandable members extends into a portion of the liner.

Alternatively or additionally to any of the embodiments above, wherein the plurality of expandable members includes a first expandable member and a second expandable member, and wherein the first expandable member is free of attachment to the second expandable member.

Alternatively or additionally to any of the embodiments above, wherein the plurality of expandable members includes a first expandable member and a second expandable member, and wherein the first expandable member is coupled to the second expandable member.

Alternatively or additionally to any of the embodiments above, wherein at least a portion of the sheath is laminated to at least a portion of the liner.

Alternatively or additionally to any of the embodiments above, wherein the sheath extends continuously around the circumference of the liner in both the first configuration and the expanded configuration.

Alternatively or additionally to any of the embodiments above, wherein the plurality if expandable members include a braided portion.

Alternatively or additionally to any of the embodiments above, wherein the introducer sheath is designed to shift between a first configuration and an expanded configuration, and wherein the introducer sheath is designed to shift from the expanded configuration back to the first configuration.

Another example introducer sheath includes:

an inner tubular member including an axial slot extending along a portion thereof, wherein the axial slot includes a first tubular wall portion on a first side of the slot and a second tubular wall portion on a second side of the slot;

a plurality of expandable members disposed along at least a portion of the axial slot, wherein the expandable members are coupled to the first tubular wall portion and the second tubular wall portion; and

an outer tubular member disposed over at least a portion of the inner tubular member and the plurality of expandable members;

wherein the introducer sheath is designed to shift between a first configuration to an expanded configuration.

Alternatively or additionally to any of the embodiments above, wherein each of the expandable members includes a first arm and a second arm, and wherein the first arm of each of the expandable members is coupled to the first tubular wall portion and the second arm of each of the expandable members is coupled to the second tubular wall portion.

Alternatively or additionally to any of the embodiments above, wherein the first arm of each of the expandable members is designed to flex away from the second arm of each of the expandable members.

Alternatively or additionally to any of the embodiments above, wherein the plurality of expandable members includes a first expandable member and a second expandable member, and wherein the first expandable member is free of attachment to the second expandable member.

Alternatively or additionally to any of the embodiments above, wherein the plurality of expandable members includes a first expandable member and a second expandable member, and wherein the first expandable member is coupled to the second expandable member.

Alternatively or additionally to any of the embodiments above, wherein at least a portion of the outer tubular member is laminated to at least a portion of the inner tubular member.

Alternatively or additionally to any of the embodiments above, wherein the outer tubular member extends continuously around the circumference of the inner tubular member in both the first configuration and the expanded configuration.

Alternatively or additionally to any of the embodiments above, wherein the plurality if expandable members include a braided portion.

Alternatively or additionally to any of the embodiments above, wherein the introducer sheath is designed to shift between a first configuration and an expanded configuration, and wherein the introducer sheath is designed to shift from the expanded configuration back to the first configuration.

An example method of treating the heart includes:

positioning an introducer sheath within a body lumen, the introducer sheath including:
a liner having a proximal portion, a distal portion and an axial slot extending along a portion thereof;
a plurality of expandable members disposed adjacent at least a portion of the axial slot; and
a sheath disposed over at least a portion of the liner and the plurality of expandable members; and

advancing a heart valve through the introducer sheath, whereby the introducer sheath expands from an unexpanded configuration to an expanded configuration to accommodate the heart valve.

Alternatively or additionally to any of the embodiments above, wherein at least a portion of each of the plurality of expandable members extends into a portion of the liner.

Alternatively or additionally to any of the embodiments above, wherein at least a portion of the sheath is laminated to at least a portion of the liner.

The above summary of some examples is not intended to describe each disclosed embodiment or every implementation of the present disclosure. The Figures, and Detailed Description, which follow, more particularly exemplify these examples.

### BRIEF DESCRIPTION OF FIGURES

FIG. 1 is a perspective view of an example introducer;

FIG. 2 is a perspective view of a component of the introducer shown in FIG. 1;

FIG. 3 is a perspective view of a component of the introducer shown in FIG. 1;

FIG. 4 is a perspective view of an example component of an introducer;

FIG. 5 is a perspective view of another example component of an introducer;

FIG. 6 is a cross-sectional view of the introducer of FIG. 1 taken along line 6-6;

FIG. 7 is a cross-sectional view of the introducer shown in FIG. 6 in an expanded configuration;

FIG. 8 is a perspective view of another example component of an introducer;

FIG. 9 is a cross-sectional view of another example introducer;

FIGS. 10-12 illustrate a medical device being inserted through an introducer.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some examples”, “other examples”, etc., indicate that the embodiment described may include one or more particular features, structures, and/or characteristics. However, such recitations do not necessarily mean that all examples include the particular features, structures, and/or characteristics. Additionally, when particular features, structures, and/or characteristics are described in connection with one embodiment, it should be understood that such features, structures, and/or characteristics may also be used connection with other examples whether or not explicitly described unless clearly stated to the contrary.

The following detailed description should be read with reference to the drawings in which similar elements in different drawings are numbered the same. The drawings, which are not necessarily to scale, depict illustrative examples and are not intended to limit the scope of the disclosure.

In some instances, performing percutaneous medical procedures may require the insertion and/or maneuvering of relatively large medical devices through a patient's vasculature. However, inserting the medical device into the vasculature may result in undesirable forces being applied to the vessel walls. For example, as the medical device passes into the vasculature, it may make undesirable contact with one or more vessel walls. This interference may cause injury to the vessel as the medical device is navigated into calcified or diseased vessels. Therefore, in some instances an introducer is utilize to facilitate the insertion of medical devices into the vessel. Further, vessel trauma resulting from forces applied to the vessel wall by a medical device may be lessened by minimizing the size of an introducer used to access the vessel. Therefore, it may be desirable to design an introducer having a reduced insertion profile, yet capable of expansion when necessary (e.g., during the passage of a medical device therethrough). The following examples disclose an intravascular medical device including an expandable introducer, whereby the introducer is designed to expand from a reduced profile, unexpanded configuration to an expanded configuration.

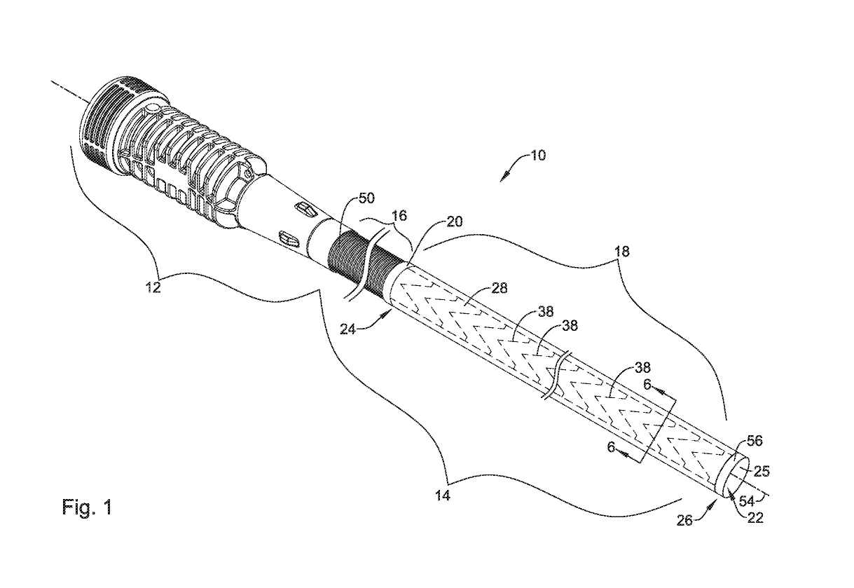

FIG. 1 illustrates an example expandable introducer (e.g., delivery sheath, access sheath, etc.) 10. The introducer 10 may include a longitudinal axis 54. Further, the introducer 10 may include a tubular member 14 attached to a hub member 12. The tubular member 14 may include a proximal section 16 and a distal section 18. The distal section 18 of the tubular member 14 may include a proximal end region 24 and a distal end region 26. The tubular member 14 may further include a lumen 22 extending therethrough. As will be described in greater detail below, an outer jacket or sheath 28 (e.g., an outer layer) may extend along the proximal section 16 and/or the distal section 18.

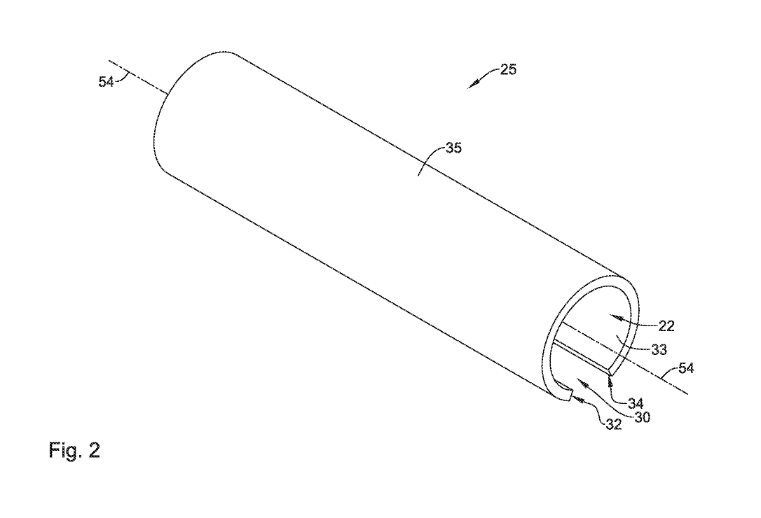

Additionally, the introducer 10 may further include an elongated compliant liner (e.g., tubular membrane, etc.) 25 (shown in greater detail in FIG. 2). The liner 25 may extend along both the distal section 18 and the proximal section 16. As illustrated in FIG. 1, the introducer 10 may also include a plurality of expandable members 38 disposed along the distal section 18. In some examples, the plurality of expandable members 38 may be aligned longitudinally along the distal section 18. As will be described in greater detail below, the outer sheath 28 and the expandable members 38 may be disposed along a portion of the liner 25 from the distal end region 26 to the proximal end region 24 of the distal section 18.

The proximal section 16 of the tubular member 14 may include a spring member 50. In some examples, an outer covering or sheath may cover either the outer surface, inner surface or both the inner and outer surfaces of the spring member 50. For example, in some instances, the spring member 50 may be positioned between (e.g., laminated) a covering positioned along the outer surface of spring member 50 and a covering positioned along the inner surface of the spring member 50.

Additionally, FIG. 1 illustrates that the expandable members 38 may terminate at a collar 20. It can further be appreciated from FIG. 1 that the collar 20 may be attached to the proximal section 16 of the tubular member 14 (e.g., the collar 20 may be attached and positioned between the distal end of proximal section 16 and the proximal end of distal section 18). Further, in some examples the collar 20 may be directly coupled to the spring member 50.

Additionally, the hub 12 may include a hemostatic valve or seal disposed therein. The hemostatic valve or seal may prevent blood or other bodily fluid(s) from flowing proximally through the lumen 22 of the tubular member 14. In at least some examples, the hub 12 may include a port in fluid communication with the lumen 22 of the tubular member 14.

In some examples it may be desirable to add a tip member to the distal end of any of the examples disclosed herein. FIG. 1 shows an example tip member 56 disposed along the distal section 18. The tip member 56 may be designed with a low durometer material and be atraumatic. In some instances, a lower durometer material may provide the tip member 56 with the ability to radially expand (e.g., flex) outward and radially contract as a variety of medical devices are advanced through the tip member 56. Further, the tip member 56 may include a taper. For example, the tip member 56 may taper from a first diameter to a second diameter at the distal end of the introducer 10. While not intended to be limiting, in some examples the shape of the tip member 56 may resemble a bull-nose. Additionally, the tip member 56 may include a radiopaque material. The radiopaque material may allow the tip member 56 to be visualized by a clinician during a medical procedure. For example, the radiopaque tip may include a radiopaque loaded polymer. In some examples, the tip member 56 may be segmented radially and/or dissected such that it may separate into segments upon expansion.

FIG. 2 illustrates an example liner 25 as described above. As shown in FIG. 2 and discussed above, the liner 25 may include a lumen 22 extending therethrough. Further, the liner 25 may include a slot 30 extending longitudinally along the liner 25. The term “slot” used herein is not intended to be limiting. In particular, the longitudinally extending “slot” 30 may also be described as a channel, slit, etc. In some instances, the slot 30 may be referred to as an axial slot 30. The axial slot 30 may extend along the length of the liner 25 substantially parallel to the longitudinal axis 54 of the introducer 10. Further, the slot 30 may extend from the distal end region 26 to the proximal end region 24 of the distal section 18 (described with respect to FIG. 1). It is further contemplated that the liner 25 may include more than one slot 30. For example, the liner 25 may include 1, 2, 3, 4, 5, 6, 7, 8 or more slots extending along the liner 25.

Additionally, FIG. 2 illustrates that the liner 25 may include an outer surface 35, an inner surface 33 and a wall thickness extending therebetween. Accordingly, it can be appreciated that slot 30 may be defined as the space spanning between a first liner wall portion 34 and a second liner wall portion 32. Further, each of the first liner wall portion 34 and the second liner wall portion 32 may define a planar surface which extends substantially perpendicular to the inner surface 33. However, it is contemplated that in some examples the first liner wall portion 34 and the second liner wall portion 32 may extend at an angle which is not perpendicular to the inner surface 33.

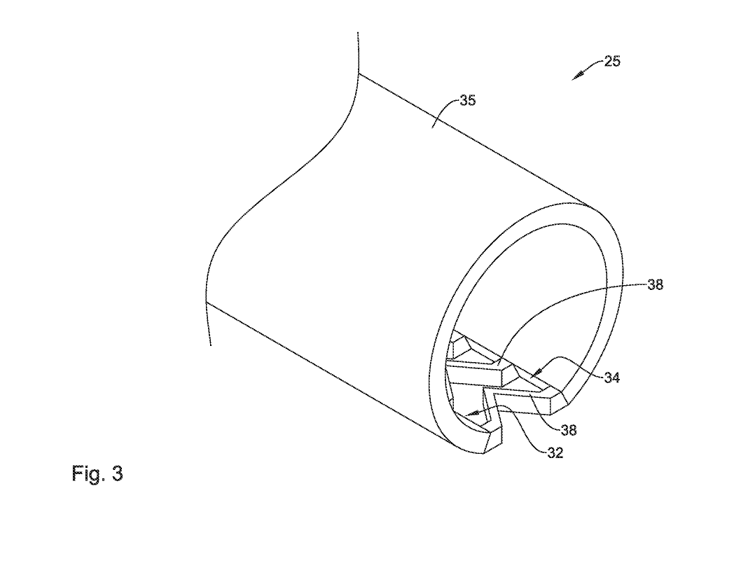

As discussed above, in some examples the plurality of expandable members 38 may be disposed along the liner 25. FIG. 3 shows one example of the plurality of expandable members 38 disposed along the liner 25. FIG. 3 illustrates that in some examples, the plurality of expandable members 38 may be disposed within the slot 30 of the liner 25. In other words, the plurality of expandable members 38 may “span” (e.g., extend from the first liner wall portion 34 to the second liner wall portion 32) the slot 30.

It can be appreciated that the plurality of expandable members 38 which span the slot 30 of the liner 25 may be attached to the liner 25 (e.g., attached to the first liner wall portion 34 and the second liner wall portion 32). For example, in some instances, a portion of each of the plurality of expandable members 38 may be fully or partially embedded into a portion of the liner 25. In other examples, a portion of each of the plurality of the expandable members 38 may be bonded to the liner 25. A variety of attachment methodologies are contemplated.

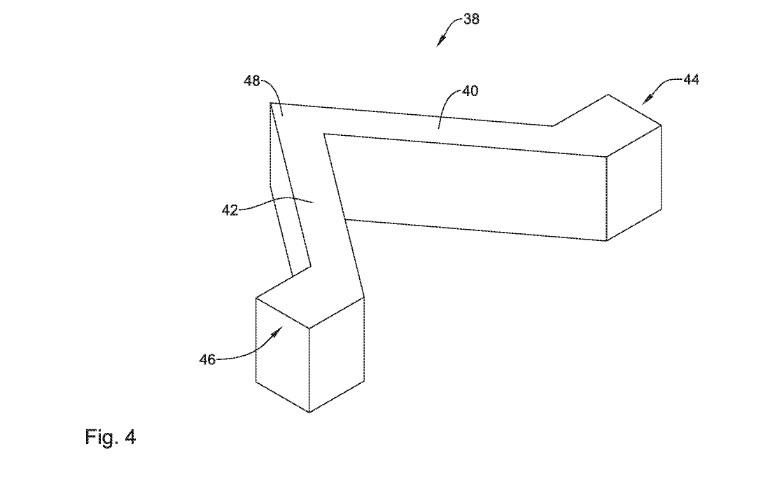

FIG. 4 illustrates an example expandable member 38. Expandable member 38 may include a first attachment point 44 and a second attachment point 46. First attachment point 44 and second attachment point 46 may define the portions of the expandable member 38 which is coupled to the liner member 25, as described above.

Further, expandable ember 38 may include a first arm 42 and a second arm 40. The first arm 40 and the second arm 42 may be attached to one another via an apex portion 48. It can be appreciated that the first arm 40 and the second arm 42 may be designed to flex away from one another. In other words, the expandable members 38 may be designed to flex and/or twist in response to external forces placed thereupon. For example, it is contemplated that the first arm 40 may be designed to flex outward or inward relative to the second arm 42. Additionally, as the first arm 40 is flexing inwardly or outwardly relative to the second arm 42, the first arm 40 may also be designed to twist out of a flat plane relative to second arm 42.

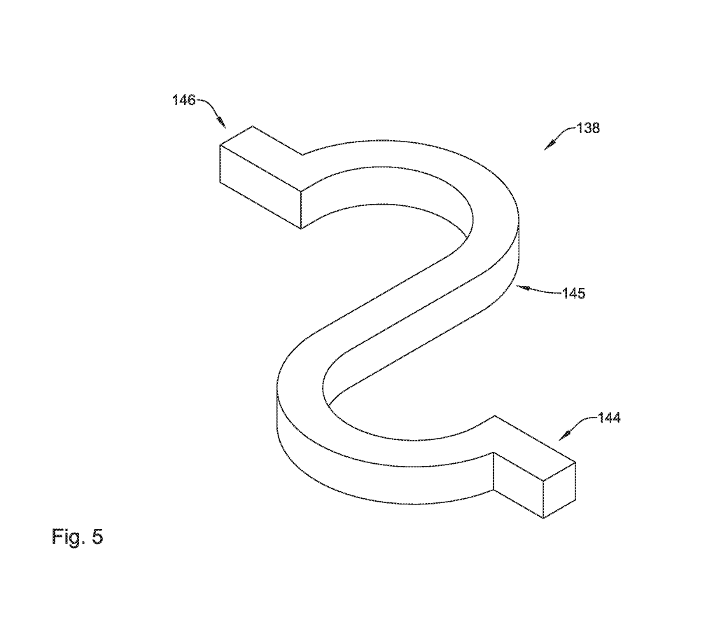

FIG. 5 illustrates another example expandable member 138. Similar to the expandable member 38 described above with respect to FIG. 3, the expandable member 138 may include a first attachment point 144 and a second attachment point 146. The first attachment point 144 and the second attachment point 146 may define the portions of the expandable member 138 which is coupled to the liner member 25, as described above. Additionally, FIG. 5 illustrates that the example expandable members 138 may include a curved portion 145. The curved portion 145 may permit the expandable member 138 to elongate and or shorten as needed. Further, the curved portion 145 may permit the expandable member 138 to twist out of a flat plane while elongating or shortening.

While the above discussion has illustrated example expandable members 38/138, other expandable members are contemplated. For example, a variety of expandable members including a variety of shapes, geometries, curves, etc. are contemplated. In some examples, the expandable members may include a braided portion. Additionally, it is contemplated that expandable members having different shapes may be utilized together in the introducer 10. For example, the expandable member 38 may be utilized with the expandable member 138. Expandable members having different shapes may be disposed in an alternating manner (or in any pattern) along the slot 30 of the liner 25, for example.

Additionally, it is contemplated that the plurality of expandable members described above may be spaced apart from one another. In other words, the expandable members described herein may not be connected to one another. It can be appreciated that the expandable members may be aligned along the longitudinal axis 54 of the introducer 50 while being separated from one another.

However, in other examples it is contemplated that the expandable members described herein may be coupled to one another. For example, it can be appreciated that each of the expandable members (e.g., the expandable members 38 shown in FIG. 4 or the expandable members 138 shown in FIG. 5) may be attached to one another. The expandable members can be attached via an elastomeric outer sleeve, elastomeric tethers radially, or tethered longitudinally.

In addition to the plurality of expandable members described above, other configurations of expandable materials are contemplated. For example, a variety of composite biomaterials defining a continuous “sheet” of expandable material is contemplated. These continuous “sheets” of material may include a variety of different fenestration patterns, including a variety of arcuate members which elastically stretch with respect to one another are contemplated herein. Some of these example sheets of materials are disclosed in U.S. Pat. No. 9,415,567, the entirety disclosure of which is herein incorporated by reference and U.S. Patent Pub. No. 2017/0007400, the entirety disclosure of which is herein incorporated by reference. In can be appreciated that a portion of these “sheets” of material illustrated in U.S. Pat. No. 9,415,567 and U.S. Patent Pub. No. 2017/0007400 may be utilized in a similar manner to the expandable members described above. For example, these expandable sheets of material may be utilized to span the slot 30 of the liner 25 described above.

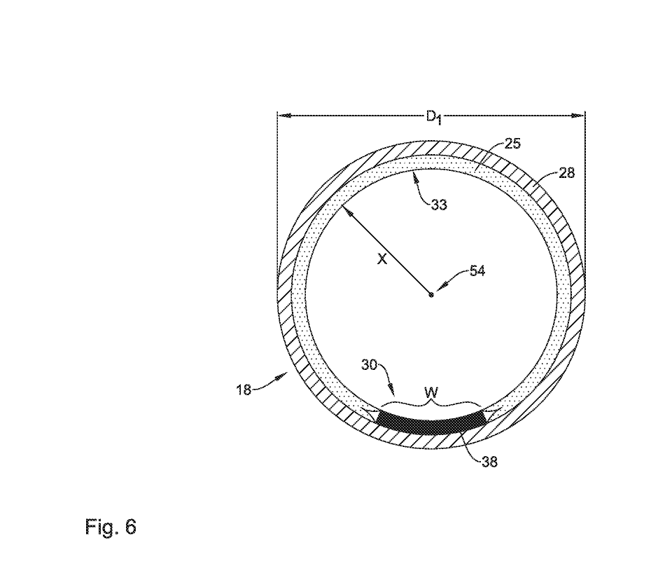

FIG. 6 shows a cross-sectional view along line 6-6 of FIG. 1. FIG. 6 illustrates a cross-section taken along the distal section 18 of the tubular member 14. As will be described in greater detail below, FIG. 6 represents a cross-section of tubular member 14 in an unexpanded configuration.

FIG. 6 further illustrates that the distal section 18 of the tubular member 14 may include the liner 25, the expandable member 38 and the outer layer (e.g., sheath, jacket, covering, etc.) 28. The liner 25 may include an inner surface 33. The distal section 18 of the tubular member 14 may include an outer diameter depicted as “D₁.” Further, the distal section 18 of the tubular member 14 may include an inner radial extent (measured from the longitudinal axis 54 of the distal section 18 to the inner surface 33 of the distal section 18) depicted as “X.”

As shown in FIG. 6, the distal section 18 may include an expandable member 38 spanning a slot 30 as described above. The expandable member 38 may be designed to flex outwardly in response to an expansion of the distal section 18. Further, FIG. 6 depicts the width of the example slot 30 as “W.” In some examples, width “W” may be about 0.25 mm to 3.0 mm.

As discussed above, FIG. 6 illustrates that the slot 30 may extend from the inner surface 33 of the liner 25 through the entire wall thickness of the liner 25. In other words, the slot 30 may extend from the inner surface 33 of the liner 25 to the inner surface of the outer layer 28. However, while FIG. 6 illustrates the slot 30 of the distal section 18 extending from the inner surface 33 of the liner 25 to the inner surface of the outer layer 28, it is contemplated that in at least some embodiments contemplated herein, the slot 30 may extend only partially into the liner 25.

Additionally, as discussed above, FIG. 6 illustrates example expandable member 38 as spanning the slot 30. In other words, in some examples the only portion of the expandable member 38 which contact the liner 25 may be the attachment points (discussed above with respect to FIG. 4). However, in other examples, it is contemplated that all or a portion of the expandable members 38 may extend into a portion of the wall thickness of the liner 25. For example, in some instances the expandable members 38 may be fully embedded in the wall thickness of the liner 25. However, in other examples, only a portion of the expandable members 38 may extend into a portion of the wall thickness of the liner 25.

Additionally, in some examples it is contemplated that the liner 25 may attached to the outer layer 28. For example, in some instances a portion of the liner 25 may be laminated, reflowed, affixed, etc. to the outer layer 28. Further, in some examples, the expandable members 38 may be laminated between the liner 25 and the outer layer 28. This may result in the expandable members 38 being coupled to the liner 25, the outer layer 28 or both the liner 25 and the outer layer 28. However, in some examples a portion of the liner 25, the outer layer 28 or both the liner 25 and the outer layer 28 may be “masked off” to prevent the expandable members 38 from being attached to either the liner 25 and/or the outer layer 28, even though portions of the liner 25 and the outer layer 28 are attached (e.g., laminated) together. This configuration may result in the expandable members 38 being positioned within a “pocket” between the liner 25 and the outer layer 28. Further, in some instances only a portion to the liner 25, the outer layer 28 or both the liner 25 and the outer layer 28 may be masked off such that a selected portion of the expandable members 38 are attached to the liner 25, the outer layer 28 or both the liner 25 and the outer layer 28. For example, the liner 25, the outer layer 28 or both the liner 25 and the outer layer 28 may be masked off such that only the attachment points (discussed above) of the expandable members 38 may be attached to the liner 25, even though portions of the liner 25 and the outer layer 28 are attached (e.g., laminated) together.

As discussed above, in some examples it may be desirable to design the introducer 10 to permit a medical device (e.g., heart valve) to pass therethrough. For example, it may be desirable to permit a medical device to pass through the hub 12, the proximal section 16 and the distal section 18 (for example, to pass through the introducer 10 while being inserted into a body lumen). Further, in some instances it may be desirable to design the introducer 10 to radially expand such that it can accommodate devices which have an outer diameter greater than the unexpanded inner diameters of the hub 12, the proximal section 16 and the distal section 18.

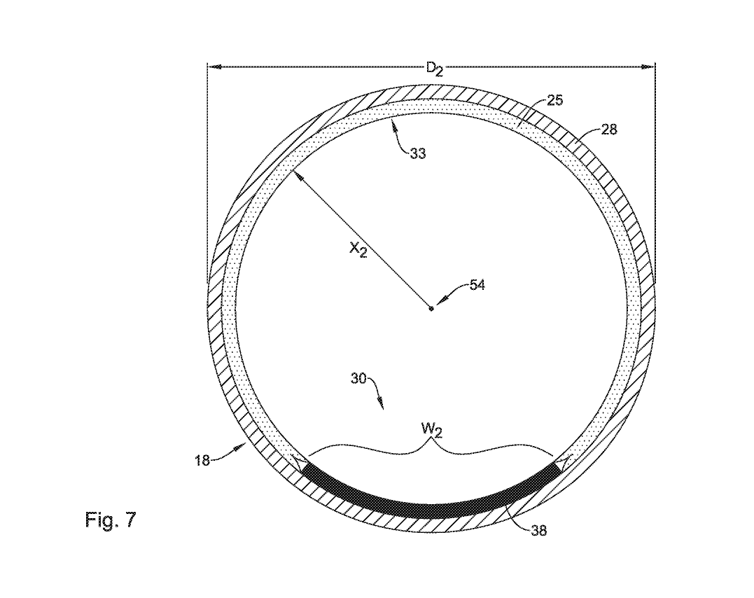

FIG. 7 represents the distal section 18 of the tubular member 14 in an expanded configuration. In other words, FIG. 7 may represent the cross-section of the tubular member 14 shown in FIG. 6 after it has been expanded radially outward.

As shown in FIG. 7, the distal section 18 of the tubular member 14 may include an outer diameter depicted as “D₂.” It can be appreciated that the expanded outer diameter D₂ may be greater than the unexpanded diameter D₁ shown in FIG. 6. In some examples, diameter “D₂” may be about 0.5% to 400% greater than diameter “D₁,” or about 1% to 300% greater than diameter “D₁,” or about 10% to 200% greater than diameter “D₁,” or about 50% to 150% greater than diameter “D₁,” or about 125% greater than diameter “D₁.” Further, the distal section 18 of the tubular member 14 may include an expanded inner radial extent (measured from the longitudinal axis 54 of the distal section 18 to the inner surface 33 of the distal section 18) depicted as “X₂.” It can be appreciated that the expanded inner radius X₂ may be greater than the unexpanded inner radius X shown in FIG. 6.

It can be appreciated that as an example introducer 10 is expanded from an unexpanded configuration to an expanded configuration (as shown in FIG. 6 and FIG. 7), the total wall thickness of the distal section 18 (including the thickness of the outer layer 28 and the liner 25) may decrease. In other words, as the distal section 18 of the tubular member 14 expands, the material defining the distal section 18 (including the thickness of the outer layer 28 and the liner 25) may stretch as the distal section 18 of the tubular member 14 expands radially outward. This stretching of the distal section 18 of the tubular member 14 may cause the wall thickness of the distal section 18 to decrease.

Additionally, it can be appreciated that as the distal section 18 of the tubular member 14 expands, the width of both the slot 30 and width of the expandable members 38 may increase. For example, FIG. 7 shows the width of the slot 30 in an expanded configuration as “W₂.” It can be appreciated that the width of the expandable members 38 may increase to substantially the same width as the slot 30. As shown, the width W₂ may be greater than W (depicted in FIG. 6). Further, as the distal section radially expands, it is contemplated that the outer layer 28 may remain continuous around the liner 25 and the expandable member 38. In other words, as the slot 30 widens (as the distal section 18 expands) the outer layer 28 may merely stretch without breaking, separating, splitting, etc. thereby maintaining a continuous outer surface around the liner 25 and the expandable members 38.

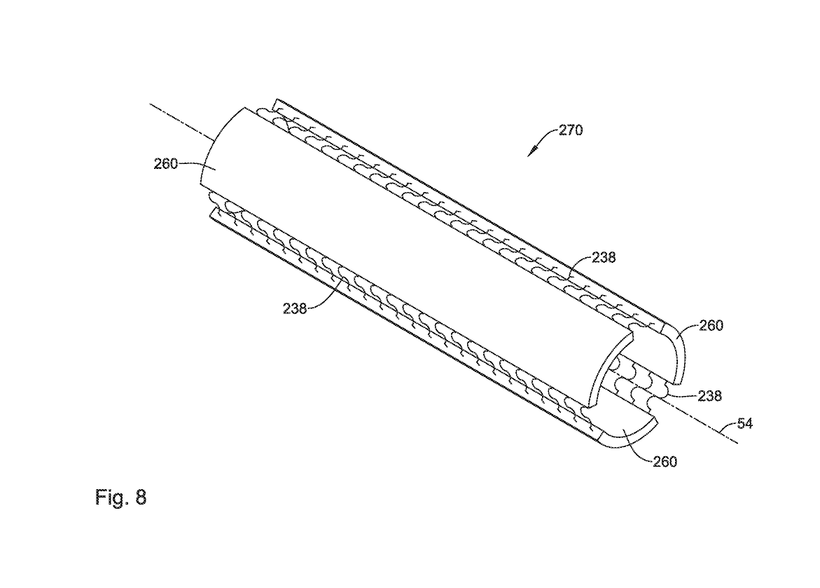

FIG. 8 illustrates another example expandable member 270. The expandable member 270 may include one or more longitudinally extending panel members 260 spaced apart from one another around the longitudinal axis 54. Further, adjacent panel members 260 may be coupled to one another by a plurality of expandable members 238. In some examples, the expandable members 238 may be similar in form and function to other expandable members described herein. The expandable member 270 may include one or more “sets” of expandable members 238 which may be, collectively, attached to two adjacent panel members 260 (as shown in FIG. 8). Further, FIG. 8 illustrates that the expandable members 238 may be longitudinally aligned with one another when positioned between two adjacent panel members 260. These longitudinally aligned expandable members 238 may operate cooperatively to permit the panel members 260 to flex radially outward with respect to the longitudinal axis 54.

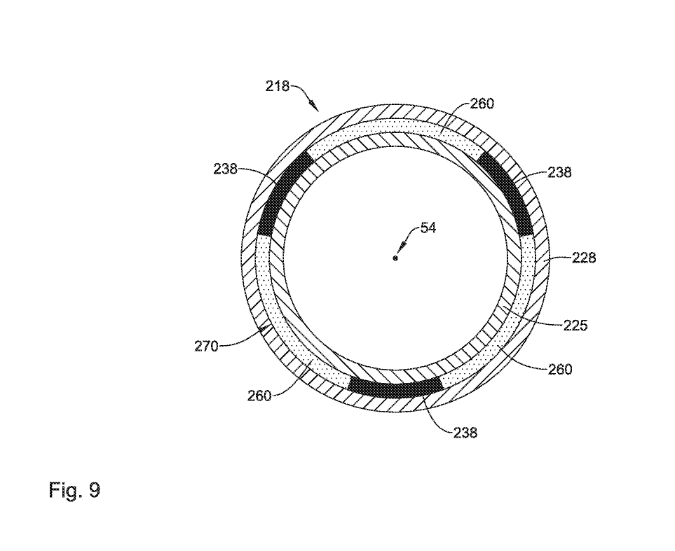

FIG. 9 shows an example cross-section of an example distal section 218 which may be similar in form and function to the distal section 18 of the introducer 10 described above. Similarly to that discussed above with respect to FIG. 1, the expandable member 270 may be incorporated in the distal section 218 of the introducer 10. It can be appreciated that when incorporated into the distal section 218 of the introducer 10, the expandable member 270 may include a liner 225 disposed along an inner surface of expandable member 270 and may also include an outer sheath 228 disposed along an outer surface of the expandable member 270. Further, FIG. 9 shows the panel members 260 spaced apart from one another around the longitudinal axis 54. Additionally, FIG. 9 illustrates the expandable members 238 positioned between (e.g., attached to) adjacent panel members 260. Further, while FIG. 9 shows three panel members 260 spaced around the longitudinal axis 54, this is not intended to be limiting. Rather, it is contemplated that the expandable member 270 may include 2, 3, 4, 5, 6, 7, 8 or more panel members 260.

Additionally, in some examples, the outer layer 228 and liner 225 shown in FIG. 9 may extend through a portion of the expandable member 270. For example, in some examples, the expandable member 270 may be “laminated” between the outer layer 228 and the liner 225. Further, the liner 225 may be radially free and delaminated from the expandable member 270 upon expansion, but remain laminated to outer layer 228.

Further, it can be appreciated that the example distal section 218 described with respect to FIG. 9 may be able to radially expand in a manner similar to the distal section 18 shown and described with respect to FIG. 6 and FIG. 7.

In some examples, the example expandable introducer 10 may be disposed about or inserted over a guidewire (not shown), although the guidewire is not required. As discussed above, in some examples the expandable introducer 10 may include a proximal section 16 and a distal section 18. In examples having a proximal section 16, the proximal section 16 may have an inner diameter or extent sufficient to accept a medical device passing therethrough, while the distal section 18 may have an inner diameter or radial extent in a relaxed condition that is less than a maximum outer diameter or extent of the medical device. The expandable introducer 10 may be formed using any of the techniques or structures discussed herein.

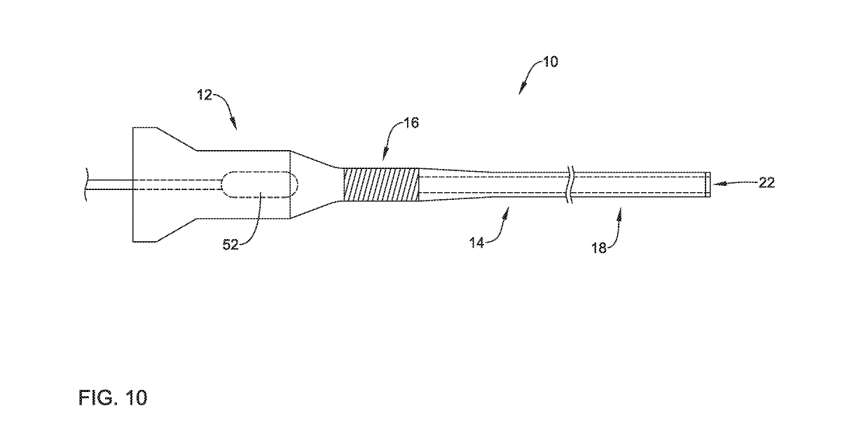

FIGS. 10-12 illustrate a method of use of introducer 10. FIG. 10 shows that an elongate medical device 52 (e.g., heart valve) may be inserted into the lumen 22 of the introducer 10 and advanced distally toward the distal end of introducer 10. As the medical device reaches, encounters, and/or engages the lumen 22 of the introducer 10, the medical device may exert a radially outward force from within the lumen 22 upon the wall of the tubular member 14. The radially outward force may cause the tubular member 14 to expand as the medical device 52 is advanced distally through the proximal section 16 and/or the distal section 18.

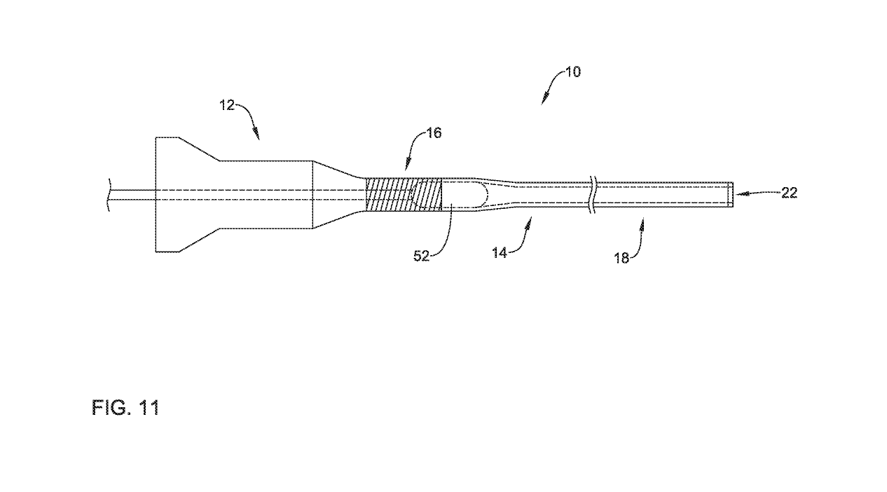

FIG. 11 illustrates the medical device 52 being inserted through proximal section 16 and into the distal section 18. As can be appreciated from FIG. 11 and the above discussion, the distal section 18 may expand radially outward as the medical device 52 is inserted therethrough.

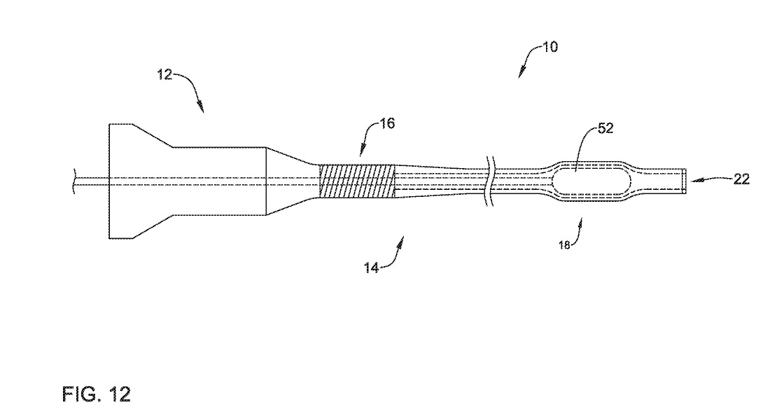

FIG. 12 illustrates the medical device 52 being positioned within the distal section 18 of introducer 10. As can be appreciated from FIG. 12, the distal section 18 may expand radially outward in order to permit medical device 52 to travel therethrough. Further, FIG. 12 illustrates distal section 18 contracting (e.g., returning) to an unexpanded diameter as the medical device 52 travels therethrough. In other words, in some examples, the distal section 18 of introducer 10 may be designed such that it can expand radially outward and then contract radially inward as medical devices are inserted therethrough. The expansion of the distal section 18 from an unexpanded configuration to an expanded configuration illustrated in FIG. 12 may correspond to the expansion from an unexpanded to an expanded configuration of the tubular members illustrated and described above with respect to FIGS. 6-9.

Further, the expansion of the distal section 18 of the medical device shown in FIG. 12 from an unexpanded configuration to an expanded configuration may be variable. For example, the diameter of the unexpanded distal section 18 of the introducer 10 may increase to an expanded diameter, after which, it may contract to a diameter that is greater than the diameter of the unexpanded configuration. However, this is not intended to be limiting. It is contemplated that once the unexpanded distal section 18 is expanded, it may remain expanded or it may return to any diameter less than the expanded diameter (including a diameter that is less than the unexpanded diameter).

In some examples, introducer 10 may be made from materials such as metals, metal alloys, polymers, ceramics, metal-polymer composites, or other suitable materials, and the like. Some examples of suitable materials may include metallic materials such as stainless steels (e.g. 304v stainless steel or 316L stainless steel), nickel-titanium alloys (e.g., nitinol, such as super elastic or linear elastic nitinol), nickel-chromium alloys, nickel-chromium-iron alloys, cobalt alloys, nickel, titanium, platinum, or alternatively, a polymeric material, such as a high performance polymer, or other suitable materials, and the like. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

In some examples, the introducer 10 may be made from materials such as, for example, a polymeric material, a ceramic, a metal, a metal alloy, a metal-polymer composite, or the like. Examples of suitable polymers may include polyurethane, a polyether-ester such as ARNITEL® available from DSM Engineering Plastics, a polyester such as HYTREL® available from DuPont, a linear low density polyethylene such as REXELL®, a polyamide such as DURETHAN® available from Bayer or CRISTAMID® available from Elf Atochem, an elastomeric polyamide, a block polyamide/ether, a polyether block amide such as PEBA available under the trade name PEBAX®, silicones, polyethylene, Marlex high-density polyethylene, polyetheretherketone (PEEK), polyimide (PI), and polyetherimide (PEI), a liquid crystal polymer (LCP) alone or blended with other materials. In some examples, a suitable polymeric material may have a yield strain of at least 20%, at least 30%, at least 40%, at least 50%, or more. In some examples, the sheath, the membrane, and/or the plurality of corrugations may be made from a material having a low coefficient of friction. In some examples, the sheath, the membrane, and/or the plurality of corrugations may be formed from a fluoropolymer, such as polytetrafluoroethylene (PTFE) or fluorinated ethylene propylene (FEP).

Portions of introducer 10 may be made of, may be doped with, may include a layer of, or otherwise may include a radiopaque material. Radiopaque materials are understood to be materials capable of producing a relatively bright image on a fluoroscopy screen or another imaging technique such as X-ray during a medical procedure. This relatively bright image aids the user of device in determining its location. For example, one or more of the elements described above (i.e., the sheath, the membrane, the medical device, etc.) may include or be formed from a radiopaque material. Suitable materials can include, but are not limited to, bismuth subcarbonate, iodine, gold, platinum, palladium, tantalum, tungsten or tungsten alloy, and the like.

It should be understood that although the above discussion was focused on percutaneous medical procedures within the vasculature of a patient, other examples or methods in accordance with the disclosure can be adapted and configured for use in other parts of the anatomy of a patient. For example, devices and methods in accordance with the disclosure can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the devices and methods described herein with respect to percutaneous deployment may be used in other types of surgical procedures as appropriate. For example, in some examples, the devices may be deployed in a non-percutaneous procedure. Devices and methods in accordance with the disclosure can also be adapted and configured for other uses within the anatomy.

It should be understood that this disclosure is, in many respects, only illustrative. Changes may be made in details, particularly in matters of shape, size, and arrangement of steps without exceeding the scope of the disclosure. The disclosure's scope is, of course, defined in the language in which the appended claims are expressed.

What is claimed is:

1. An introducer sheath, comprising:
a liner having a proximal portion, a distal portion and an axial slot extending along a portion thereof;
a plurality of expandable members disposed adjacent at least a portion of the axial slot; and
a sheath disposed over at least a portion of the liner and the plurality of expandable members;
wherein the introducer sheath is designed to shift between a first configuration and an expanded configuration.

2. The introducer of claim 1, wherein at least one of the plurality of expandable members extends into a portion of the liner.

3. The introducer of claim 1, wherein the plurality of expandable members includes a first expandable member and a second expandable member, and wherein the first expandable member is free of attachment to the second expandable member.

4. The introducer of claim 1, wherein the plurality of expandable members includes a first expandable member and a second expandable member, and wherein the first expandable member is coupled to the second expandable member.

5. The introducer of claim 1, wherein at least a portion of the sheath is laminated to at least a portion of the liner.

6. The introducer of claim 1, wherein the sheath extends continuously around the circumference of the liner in both the first configuration and the expanded configuration.

7. The introducer of claim 1, wherein the plurality if expandable members include a braided portion.

8. The introducer of claim 1, wherein the introducer sheath is designed to shift between a first configuration and an expanded configuration, and wherein the introducer sheath is designed to shift from the expanded configuration back to the first configuration.

9. An introducer sheath, comprising:
an inner tubular member including an axial slot extending along a portion thereof, wherein the axial slot includes a first tubular wall portion on a first side of the slot and a second tubular wall portion on a second side of the slot;
a plurality of expandable members disposed along at least a portion of the axial slot, wherein the expandable members are coupled to the first tubular wall portion and the second tubular wall portion; and
an outer tubular member disposed over at least a portion of the inner tubular member and the plurality of expandable members;
wherein the introducer sheath is designed to shift between a first configuration to an expanded configuration.

10. The introducer of claim 9, wherein each of the expandable members includes a first arm and a second arm, and wherein the first arm of each of the expandable members is coupled to the first tubular wall portion and the second arm of each of the expandable members is coupled to the second tubular wall portion.

11. The introducer of claim 9, wherein the first arm of each of the expandable members is designed to flex away from the second arm of each of the expandable members.

12. The introducer of claim 9, wherein the plurality of expandable members includes a first expandable member and a second expandable member, and wherein the first expandable member is free of attachment to the second expandable member.

13. The introducer of claim 9, wherein the plurality of expandable members includes a first expandable member and a second expandable member, and wherein the first expandable member is coupled to the second expandable member.

14. The introducer of claim 9, wherein at least a portion of the outer tubular member is laminated to at least a portion of the inner tubular member.

15. The introducer of claim 9, wherein the outer tubular member extends continuously around the circumference of the inner tubular member in both the first configuration and the expanded configuration.

16. The introducer of claim 9, wherein the plurality if expandable members include a braided portion.

17. The introducer of claim 9, wherein the introducer sheath is designed to shift between a first configuration and an expanded configuration, and wherein the introducer sheath is designed to shift from the expanded configuration back to the first configuration.

18. A method of treating the heart, the method comprising:
positioning an introducer sheath within a body lumen, the introducer sheath including:
a liner having a proximal portion, a distal portion and an axial slot extending along a portion thereof;
a plurality of expandable members disposed adjacent at least a portion of the axial slot; and
a sheath disposed over at least a portion of the liner and the plurality of expandable members; and
advancing a heart valve through the introducer sheath, whereby the introducer sheath expands from an unexpanded configuration to an expanded configuration to accommodate the heart valve.

19. The method of claim 18, wherein at least a portion of each of the plurality of expandable members extends into a portion of the liner.

20. The method of claim 18, wherein at least a portion of the sheath is laminated to at least a portion of the liner.
